(19) United States
(12) Patent Application Publication
(10) Pub. No.: US 2022/0212037 A1
(43) Pub. Date: Jul. 7, 2022

(54) RADIOTHERAPY DEVICE AND RADIOTHERAPY METHOD

(71) Applicant: SUZHOU LINATECH MEDICAL SCIENCE AND TECHNOLOGY CO.,LTD., Jiangsu (CN)

(72) Inventors: Hu Er WEN, Jiangsu (CN); Jonathan Yi YAO, Jiangsu (CN)

(21) Appl. No.: 17/605,467

(22) PCT Filed: Nov. 7, 2019
(86) PCT No.: PCT/CN2019/116429

§ 371 Date: Oct. 21, 2021

(30) Foreign Application Priority Data
Apr. 22, 2019 (CN) 201910326332.1
Apr. 22, 2019 (CN) 201920553757.1

(51) Int. Cl. A61N 5/10

(52) U.S. Cl. CPC A61N 5/1081; A61N 5/1084; A61N 2005/1059; A61N 2005/1087; A61N 5/1049

(57) ABSTRACT

The invention discloses a radiotherapy device and method, the invention uses the treatment reference point of the photon therapy component, that is, the isocenter as the reference point of the entire system, so that the axis of the proton beam emitted by the proton treatment equipment (proton therapy component) or the heavy ion beam emitted by the heavy ion treatment equipment (heavy ion therapy component) always passes through the reference point, then the two can simultaneously or separately treat patients who have successfully placed one time. The treatment plan can be optimized, and the respective treatment systems of the two treatment systems can be combined. Advantages, to complete the treatment together, especially for some complex cases, the combination of the two can better complete the treatment task at a lower cost.

### TECHNICAL FIELD

The invention relates to a radiotherapy device, in particular to a radiotherapy device and method.

### BACKGROUND

With the development of tumor radiology and material science, high-energy photon therapy devices, proton therapy devices, and heavy ion therapy devices are all undergoing continuous development. These three technical solutions each have their own advantages and disadvantages. In some cases, a variety of treatment plans are needed to obtain better treatment results, and patients need to complete the positioning and treatment processes in different treatment rooms. This not only extends the treatment time, but also increases the links, repeats of positioning and medical fee. There is no plan to integrate the above-mentioned treatment systems.

### SUMMARY

In order to solve the above-mentioned problems, the purpose of the present invention is to provide a radiotherapy device and method that can be configured with a photon therapy component and a proton therapy component at the same time, or a photon therapy component and a heavy ion therapy component can be configured at the same time, so as to overcome the drawbacks of the prior art.

To achieve the above objective, the technical solution of the present invention is:

A radiotherapy device includes:

a photon therapy component, the photon therapy component includes a bracket, a gantry and an accelerator, the gantry is rotatably mounted on the bracket, and the accelerator is installed on the gantry; the gantry drives the accelerator to rotate around the isocenter, and the axis of the photon beam emitted by the accelerator always passes through a reference point: the isocenter;

a proton therapy component or a heavy ion therapy component, the proton therapy component is used to generate a proton beam for treatment; the heavy ion treatment component is used to generate a heavy ion beam for treatment;

the axis of the proton beam emitted by the proton therapy component or the heavy ion beam emitted by the heavy ion therapy component always passes through the isocenter.

At present, proton therapy equipment (proton therapy component) or heavy ion therapy equipment (heavy ion therapy component) is relatively large, while photon therapy equipment (photon therapy component, such as X-ray radiotherapy equipment) has been miniaturized, and an accelerator can be installed on a rotatable gantry, it is realized that the ray beam can be emitted from different points on a ring (or even a part of the spherical surface) to the isocenter, that is, the incident angle of the ray beam of the photon treatment equipment can be adjusted within a certain range. However, proton therapy equipment and heavy ion therapy equipment are far from being miniaturized, and their proton beams and heavy ion beams are generally only emitted along a fixed axis, and the incident angle cannot be adjusted in real time. Therefore, there are many difficulties in combining the photon therapy equipment with the two. The present invention uses the treatment reference point of the photon therapy device, that is, the isocenter as the reference point of the entire system, so that the proton therapy device (proton therapy component) or the heavy ion therapy device (Heavy ion therapy component) The axis of the emitted proton beam or heavy ion beam always passes through the reference point. Then, both can treat patients who are successfully positioned at the same time or separately. The treatment plan can be optimized and the two treatments can be combined. The respective advantages of the system can be used to complete the treatment together, especially for some complex cases, the combination of the two can better complete the treatment task at a lower cost.

Further, the axis of the proton beam emitted by the proton therapy component or the heavy ion beam emitted by the heavy ion therapy component is parallel to the horizontal line. In practical applications, in order to facilitate the positioning of the patient, the axis of the proton beam emitted by the proton therapy component or the heavy ion beam emitted by the heavy ion therapy component is parallel to the horizontal line, in this way, although the emission axis of the proton beam or heavy ion beam is fixed, it is also convenient to change the inclination and rotation angle of the treatment bed in multiple dimensions to obtain the best incident angle of the proton beam or heavy ion beam. At the same time, the accelerator of the photon therapy component rotates on an arc centered on the isocenter. (Some accelerators can adjust the inclination angle, that is, the spherical knife system moves on the spherical surface with the isocenter as the center of the sphere.) There is no interference between the two. The incident angle and launch timing of the photon, and the multi-dimensional angle of the treatment bed can be Organically optimized and integrated with the launch timing of the proton beam or heavy ion beam, so that the two treatment options can be organically combined.

Further, the axis of the proton beam emitted by the proton therapy component or the heavy ion beam emitted by the heavy ion therapy component is fixed.

Further, the gantry is provided with a window through which the proton beam emitted by the proton therapy component or the heavy ion beam emitted by the heavy ion therapy component passes. Generally speaking, the axis of rotation of the photon therapy component is a horizontal line passing through the isocenter. The plane of the photon beam is perpendicular to the horizontal line. When the photon therapy component is pitching (spherical motion), the plane where the photon beam is located It is at a non-ninety degree angle with the horizontal line. In order to optimize the structure of the entire system in the treatment room, the proton beam or heavy ion beam is most suitable to be emitted along this horizontal line. It is even better that the treatment bed is located on the front of the photon therapy component. The exit point of the proton therapy component or heavy ion therapy component is located on the back of the photon therapy component so that the proton beam or heavy ion beam passes through the photon therapy component and reaches the isocenter along the above horizontal line. Here, it is set up on the main body of the photon therapy component. There is a window through which the proton beam emitted by the proton therapy component or the heavy ion beam emitted by the heavy ion therapy component passes, so that no matter how the photon therapy component operates, its structural components will not hinder the passage of the proton beam or heavy ion beam. Similarly, such a layout also makes the proton therapy component or heavy ion therapy component not affect the operation of the photon therapy component, and will not occupy other space, thereby not hindering the work of other equipment or staff.

Further, the axis of the proton beam emitted by the proton therapy component or the heavy ion beam emitted by the heavy ion therapy component is on the same plane as the axis of rotation of the accelerator.

The invention also provides a radiation therapy method, which simultaneously configures a photon therapy component and a proton therapy component, or simultaneously configures a photon therapy component and a heavy ion therapy component, comprising the following steps:

First, take the isocenter of the photon therapy component as the reference point for treatment;

Second, the axis of the proton beam emitted by the proton therapy component or the heavy ion beam emitted by the heavy ion therapy component always passes through the reference point;

Third, position the patient based on the reference point; Fourth, use photon therapy components and/or proton therapy components, or use photon therapy components and/or heavy ion therapy components to perform radiotherapy on patients.

Further, the axis of the proton beam emitted by the proton therapy component or the heavy ion beam emitted by the heavy ion therapy component is parallel to the horizontal line.

Further, the axis of the proton beam emitted by the proton therapy component or the heavy ion beam emitted by the heavy ion therapy component is fixed.

Further, the gantry is provided with a window through which the proton beam emitted by the proton therapy component or the heavy ion beam emitted by the heavy ion therapy component passes.

Further, the axis of the proton beam emitted by the proton therapy component or the heavy ion beam emitted by the heavy ion therapy component is on the same plane as the axis of rotation of the accelerator.

### BRIEF DESCRIPTION OF THE DRAWINGS

FIGS. 1 and 2 are schematic diagrams of the present invention;

the names of the corresponding parts are indicated by the numbers and letters in the figure:
1. support; 11. gantry; 12. accelerator; 13. photon beam axis; 14. window;
2. proton therapy component or heavy ion therapy component; 21. proton beam or heavy ion beam output part; 22. proton beam or heavy ion beam axis;
3. isocenter;
4. treatment bed.

### DETAILED DESCRIPTION OF THE EMBODIMENTS

The present invention will be further described in detail below in conjunction with the drawings and specific embodiments.

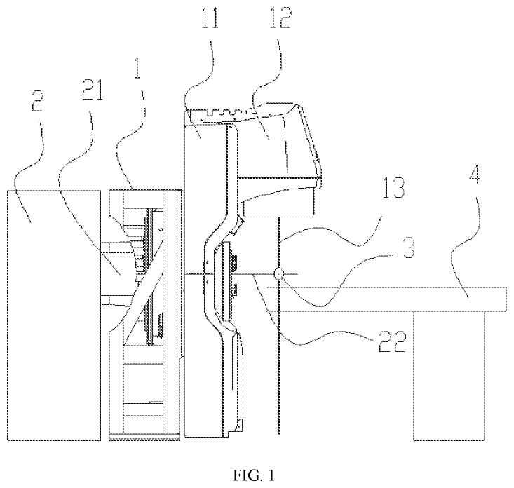

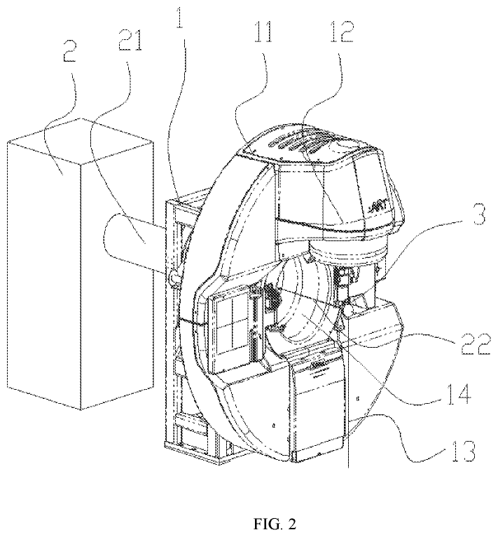

An embodiment of the present invention, as shown in FIG. 1 and FIG. 2, in order to achieve the above objective, the technical solution of the present invention is:

A radiotherapy device includes:

a photon therapy component, the photon therapy component includes: a support 1, a gantry 11 and an accelerator 12, the gantry 11 is rotatably mounted on the support 1, the accelerator 12 is installed on the gantry 11; the gantry 11 drives the accelerator 12 to rotate around the isocenter 3, the axis 13 of the photon beam emitted by the accelerator 12 always passes through a reference point: isocenter 3;

a proton therapy component or a heavy ion therapy component 2, the proton therapy component is used to generate a proton beam for treatment; the heavy ion treatment component is used to generate a heavy ion beam for treatment;

The axis 22 of the proton beam emitted by the proton therapy component or the heavy ion beam emitted by the heavy ion therapy component always passes through the isocenter 3.

At present, proton therapy equipment (proton therapy component) or heavy ion therapy equipment (heavy ion therapy component) is relatively large, while photon therapy equipment (photon therapy component, such as X-ray radiotherapy equipment) has been miniaturized, and an accelerator can be installed on a rotatable gantry, it is realized that the ray beam can be emitted from different points on a ring (or even a part of the spherical surface) to the isocenter, that is, the incident angle of the ray beam of the photon treatment equipment can be adjusted within a certain range. However, proton therapy equipment and heavy ion therapy equipment are far from being miniaturized, and their proton beams and heavy ion beams are generally only emitted along a fixed axis, and the incident angle cannot be adjusted in real time. Therefore, there are many difficulties in combining the photon therapy equipment with the two. The present invention uses the treatment reference point of the photon therapy device, that is, the isocenter as the reference point of the entire system, so that the axis of the emitted proton beam or heavy ion beam always passes through the reference point for proton therapy device (proton therapy component) or the heavy ion therapy device (heavy ion therapy component). Then, both can treat patients who are successfully positioned at the same time or separately. The treatment plan can be optimized and the two treatments can be combined. The respective advantages of the system can be used to complete the treatment together, especially for some complex cases, the combination of the two can better complete the treatment task at a lower cost.

In practical applications, because the proton beam or heavy ion beam cannot change the exit angle during use, but in order to make it cooperate with the photon therapy component, the axis of the proton beam or heavy ion beam must pass through the isocenter. There are many selected exit angles, but considering the linkage with the treatment bed and the cooperation with the photon therapy component, the axis of the proton beam emitted by the proton therapy component or the heavy ion beam emitted by the heavy ion therapy component is parallel to the horizontal line. In practical applications, in order to facilitate the positioning of the patient, the axis of the proton beam emitted by the proton therapy component or the heavy ion beam emitted by the heavy ion therapy component is parallel to the horizontal line, in this way, although the emission axis of the proton beam or heavy ion beam is fixed, it is also convenient to change the inclination and rotation angle of the treatment bed in multiple dimensions to obtain the best incident angle of the proton beam or heavy ion beam. At the same time, the accelerator of the photon therapy component rotates on an arc centered on the isocenter. (Some accelerators can adjust the inclination angle, that is, the spherical knife system moves on the spherical surface with the isocenter as the center of the sphere.) There is no interference between the two. The incident angle and launch timing of the photon, and the multi-dimensional angle of the treatment bed can be Organically optimized and integrated with the launch timing of the proton beam or heavy ion beam, so that the two treatment options can be organically combined. Furthermore, the axis of the proton beam emitted by the proton therapy component or the heavy ion beam emitted by the heavy ion therapy component is horizontal and fixed.

Further, the gantry is provided with a window 14 through which the proton beam emitted by the proton therapy component or the heavy ion beam emitted by the heavy ion therapy component passes. Generally speaking, the axis of rotation of the photon therapy component is a horizontal line passing through the isocenter. The plane where the photon beam located is perpendicular to the horizontal line. When the photon therapy component is pitching (spherical motion), the plane where the photon beam is located is at a non-ninety degree angle with the horizontal line. In order to optimize the structure of the entire system in the treatment room, the proton beam or heavy ion beam is most suitable to be emitted along this horizontal line. It is even better that the treatment bed is located on the front of the photon therapy component. The exit point of the proton therapy component or heavy ion therapy component is on the back of the photon therapy component, as shown in FIGS. 1 and 2, so that the proton beam or heavy ion beam passes through the photon therapy component and reaches the isocenter along the above horizontal line. Here the gantry of the photon therapy component is provided with a window through which the proton beam emitted by the proton therapy component or the heavy ion beam emitted by the heavy ion therapy component passes, so that no matter how the photon therapy component operates, its structural components will not affect the proton beam or obstruct the passage of heavy ion beams. Similarly, such a layout also makes the proton therapy component or heavy ion therapy component not affect the operation of the photon therapy component, and will not occupy other space, thereby not hindering the work of other equipment or staff. The window 14 described here can be a through physical through hole, or it can be a part of the path through which the proton beam or heavy ion beam passes, and will not be hinder or effect the treatment. It also can be a combination of substances that will not hinder the treatment effect, and physical cavities.

The treatment bed 4 in FIG. 1 can be a traditional style, a structure supported by a mechanical arm that can be adjusted in multiple dimensions, or a seat or a structure that can be converted between a flat panel and a seat.

The photon therapy device (photon therapy component), proton therapy device (proton therapy component), and heavy ion therapy device (heavy ion therapy component) are mostly involved in the prior art, so we will not repeat them here.

Further, the axis of the proton beam emitted by the proton therapy component or the heavy ion beam emitted by the heavy ion therapy component is on the same plane as the axis of rotation of the accelerator.

The present invention also provides a radiotherapy method, which includes the following steps:

First, take the isocenter of the photon therapy component as the reference point for treatment;

Second, the axis of the proton beam emitted by the proton therapy component or the heavy ion beam emitted by the heavy ion therapy component always passes through the reference point;

Third, position the patient based on the reference point; Fourth, use photon therapy component and/or proton therapy component, or use photon therapy component and/or heavy ion therapy component to perform radiotherapy on patients.

Further, the axis of the proton beam emitted by the proton therapy component or the heavy ion beam emitted by the heavy ion therapy component is parallel to the horizontal line.

Further, the axis of the proton beam emitted by the proton therapy component or the heavy ion beam emitted by the heavy ion therapy component is fixed.

Further, the gantry is provided with a window through which the proton beam emitted by the proton therapy component or the heavy ion beam emitted by the heavy ion therapy component passes.

Further, the axis of the proton beam emitted by the proton therapy component or the heavy ion beam emitted by the heavy ion therapy component is on the same plane as the rotation axis of the accelerator.

The above are only the preferred embodiments of the present invention. It should be pointed out that for those of ordinary skill in the art, without departing from the inventive concept of the present invention, a number of modifications and improvements can be made, all of which within the scope of protection of the invention.

1. A radiotherapy device, comprising:
a photon therapy component, the photon therapy component includes a bracket, a gantry and an accelerator, the gantry is rotatably mounted on the bracket, and the accelerator is installed on the gantry; the gantry drives the accelerator to rotate around the isocenter, and the axis of the photon beam emitted by the accelerator always passes through a reference point: the isocenter;
a proton therapy component or a heavy ion therapy component, the proton therapy component is used to generate a proton beam for treatment; the heavy ion treatment component is used to generate a heavy ion beam for treatment, including an ion source, an accelerator, and an ion scanner; the axis of the proton beam emitted by the proton therapy component or the heavy ion beam emitted by the heavy ion therapy component always converges on the isocenter.

2. The radiotherapy device according to claim 1, wherein the proton beam emitted by the proton therapy component or the heavy ion beam emitted by the heavy ion therapy component can be irradiated with the photon beam simultaneously or in time sharing.

3. The radiotherapy device according to claim 2, wherein the proton beam emitted by the proton therapy component or the heavy ion beam emitted by the heavy ion therapy component shares a set of patient bed, patient fixing device and image guiding device with the photon beam.

4. The radiotherapy device according to claim 1, wherein the gantry is provided with a window through which the proton beam emitted by the proton therapy component or the heavy ion beam emitted by the heavy ion therapy component passes.

5. The radiotherapy device according to claim 4, wherein the axis of the proton beam emitted by the proton therapy component or the heavy ion beam emitted by the heavy ion therapy component is on the same plane as the axis of rotation of the accelerator.

6. A radiotherapy method, the photon therapy component and the proton therapy component are configured at the same time, or the photon therapy component and the heavy ion therapy component are configured at the same time, including the following steps:
first, take the isocenter of the photon therapy component as the reference point for treatment;
second the axis of the proton beam emitted by the proton therapy component or the heavy ion beam emitted by the heavy ion therapy component always passes through the reference point;
third, position the patient based on the reference point;
fourth, use the photon therapy component and/or proton therapy component, or use the photon therapy component and/or heavy ion therapy component to perform simultaneous radiotherapy on patients.

7. The radiotherapy method according to claim 6, wherein the axis of the proton beam emitted by the proton therapy component or the heavy ion beam emitted by the heavy ion therapy component is parallel to the horizontal line.

8. The radiotherapy method according to claim 7, wherein the axis of the proton beam emitted by the proton therapy component or the heavy ion beam emitted by the heavy ion therapy component is fixed.

9. The radiotherapy method according to claim 6, wherein the gantry is provided with a window through which the proton beam emitted by the proton therapy component or the heavy ion beam emitted by the heavy ion therapy component passes.

10. The radiotherapy method according to claim 9, wherein the axis of the proton beam emitted by the proton therapy component or the heavy ion beam emitted by the heavy ion therapy component is on the same plane as the axis of rotation of the accelerator.
